(19) United States
(12) Patent Application Publication
(10) Pub. No.: US 2014/0058319 A1
(43) Pub. Date: Feb. 27, 2014

(54) Compact Device For Rapidly Mixing and Delivering Substances To a Patient

(71) Applicant: GLUCAGO LLC, El Segundo, CA (US)

(72) Inventors: Rush L. BARTLETT, II, Palo Alto, CA (US); Peter M. GRECO, Jr., Hermosa Beach, CA (US); Zachary R. GOSNELL, Terre Haute, IN (US); Jared L. WEST, Liberty Township, OH (US); Barry J. DAVIGNON, Terre Haute, IN (US); Arthur L. CHlEBOWSKI, Granger, IN (US)

(73) Assignee: GLUCAGO LLC, El Segundo, CA (US)

(21) Appl. No.: 14/072,607

(22) Filed: Nov. 5, 2013

Related U.S. Application Data
(63) Continuation of application No. 13/255,753, filed on Mar. 20, 2012.

(51) Int. Cl. A61M 5/19

(52) U.S. Cl. CPC A61M 5/19
USPC 604/87

(57) ABSTRACT

Injection devices are disclosed for injections of substances. The disclosed devices separately may hold two separate substances, e.g., solid, liquid or gas substances, in separate compartments which are interrupted so that the separate substances come into contact during the activation of the device. One disclosed device includes a needle for injection and two substances that are mixed with only a few actions by the operator of the device. The device may or may not activate mixing by the removal of a sleeve which triggers compartment interaction through movement of pins, pegs, springs, wires, string, or other mechanisms which allow the materials held in separate chambers to come into contact with each other. Operator error during mixing and checking the dosage of substances to be injected is avoided with the disclosed devices. Also, the needle is maintained in a sterile state because the needle is held internally of the device until the sleeve is removed and the device begins to activate.

### CROSS-REFERENCE TO RELATED APPLICATION(S)

This application is a continuation application of Ser. No. 13/255,753, filed Mar. 20, 2012, which claims the benefit of U.S. national phase filing of International Application No. PCT/US10/026670 filed Mar. 9, 2010, and which claims priority to U.S. Provisional Patent Application Nos. 61/241,462, filed on Sep. 11, 2009; 61,182,835, filed on Jun. 1, 2009; and 61/158,435, filed on Mar. 9, 2009. The disclosures of all the above-referenced patent applications are hereby fully incorporated by reference.

### BACKGROUND

1. Technical Field

Devices are disclosed for rapidly mixing and delivering substances, such a lyophilized or spray dried substances. For example, devices are disclosed for rapidly mixing and injecting solid stored substances, such as glucagon, vaccines, animal products, combat medications, antibodies, recombinant proteins, anti-venoms, vitamins, drugs, compounds, and many others, for use in many different types of therapies and treatments are possible. Methods of rapidly treating hypoglycemia and other conditions are also disclosed.

1. Description of the Related Art

Diabetes mellitus affects over 24 million Americans and is the seventh leading cause of death nationally. Approximately 17.9 million diabetics have been diagnosed in the United States. Diabetes can take two forms: Types I and II. Type I patients cannot produce insulin while Type II patients suffer from impaired glucose regulatory pathways or insulin production. Both Types I and II patients require treatment with insulin injections, lifestyle changes, monitoring, or oral medications.

Hypoglycemia is a condition of lower than normal blood sugar that results in a lifestyle of a diabetic patient. Hypoglycemia can result in coma, seizure, or even death in 2-4% of diabetics. Decreased levels of glucose can have damaging effects on the brain or other organs. The incidence of hypoglycemia in the diabetic population is hard to measure because so many the level of hypoglycemia amongst diabetics varies. Usually, a type I diabetic experiences a severe hypoglycemia episode at least once per year and mild episodes several times per week, Type II diabetics have a 20% chance of having a severe attack sometime during their life but also have mild episodes in higher frequencies. A blood glucose level below 50 mg/dL is considered a severe episode of hypoglycemia. However, as patients age, some lose their ability to recognize symptoms.

Diabetics currently only have two options to treat severe hypoglycemia. Both options are hypoglycemic rescue kits manufactured by Nova Nordisk and Eli Lilly. These kits comprise a single needle filled with a sterile solution, and a vial of glucagon/lactose powder which is mixed to and injected into the patient by hand. This method is dangerous and not effective for a wide number of potential users who are unfamiliar with needles, or have trouble focusing on the need to mix the powder and liquid during the stress of an attack on themselves or a person whom they are assisting in an emergency.

Accordingly, improved devices and methods for conveniently and quickly delivering medical products to a patient are needed.

### SUMMARY OF THE DISCLOSURE

Injection devices are disclosed that mix and inject substances such as, but not limited to, glucagon, epinephrine, anti-venoms, heart attack rescue reagents, drugs, solid compounds, powdered compounds, liquid compounds, antibodies, vitamins, nucleic acids, proteins, peptides, etc. The disclosed devices separately may hold two separate substances, e.g., solid, liquid or gas substances, in separate compartments which are interrupted so that the separate substances come into contact during the activation of the device. One disclosed device includes a needle for injection and two substances that are mixed with only a few actions by the operator of the device. The device may or may not activate mixing by the removal of a sleeve which triggers compartment interaction through movement of pins, pegs, springs, wires, string, or other mechanisms which allow the materials held in separate chambers to come into contact with each other.

Operator error during mixing and checking the dosage of substances to be injected is avoided with the disclosed devices. Also, the needle is maintained in a sterile state because the needle is held internally of the device until the sleeve is removed and the device begins to activate.

The disclosed devices can be used to mix and inject two or more substances into a patient or inject a single substance without mixing. The substances may be solid, liquid or gas substances and can be used for, but not limited to, injection of vaccines, proteins, peptides, vitamins, drugs, substances, compounds, nano particles, devices, etc. The disclosed devices will allow simple administration with minimal interaction on the part of the user taken place suggested but not limited to the removal of the sleeve of the device and then compression at the desired site of injection causing the mechanism of action to mix and inject the substances. The removal of the sleeve may cause the mixing as illustrated in three of the designs below, or the sleeve may merely protect the mechanism from preliminary injection or unsterile contamination.

As a general example, for the case of glucagon injection, glucagon will be held in a lyophilized form in one compartment. Then the sleeve of the device is removed and the device is used by the patient causing the mixing of the glucagon with a solvent and then subsequent injection into the patient there by raising the blood glucose level of the patient as a possible treatment of a hypoglycemic attack. The mechanisms may or may not be designed as to indicate an expired device, a used device, a malfunctioning device, or may or may not also retract the needle into the device as to cover it from secondary puncture after use. The advantages of separate chambers for the substances increase the shelf life of the substances.

In one design, a disclosed device comprises a sleeve which is removed prior to use causing a thin component to be pulled through the device to activate mixing of the substances to be injected. Then the sleeve is fully removed and the device is ready for compression. In the front housing of the device the front of a needle is positioned between two membranes which hold a liquid reservoir in front of the needle as to help keep the needle primed with liquid for use prior to injection into the skin. Also, the device may inject without having the extra need for a shell because the device locks and then moves to the inner component of the device to serve as an activating mechanism for the plunger which then helps to expel the contents of the device out the end of the needle and into a patient. Once the device is used it may or may not indicate that it has been used and it may or may not have a spring in the front compartment of the device or other mechanism elsewhere which causes the needle to be retracted all or partially into the housing of the device as to help prevent secondary injury from the exposed needle.

In one embodiment, a disclosed device for mixing at least two substances to form a mixture and for delivering the mixture to a patient comprises a first chamber in communication with a dispense outlet. The first chamber accommodates a first substance and a second chamber accommodates a second substance. The second chamber is isolated from the first chamber by a barrier. The barrier coupled to a sleeve. Wherein, movement of the sleeve under manual pressure causes the barrier to become dislodged and the first and second substances to mix in at least one of the first and second chambers.

In a refinement, the dispense outlet is a cannula.

In a refinement, the second substance is a powder.

In a refinement, the first substance is a liquid.

In a refinement, the first substance of the first chamber is a liquid of the second substance of the second chamber is a powder.

In a refinement, the first chamber is disposed between the sleeve and the second chamber.

In a refinement, the second chamber is disposed between the upper housing and the barrier,

In a refinement, the barrier is a piece of foil or material that is easily torn, broken, or moved.

In a refinement, the barrier may coupled to the sleeve by a coupling element that passes through the cannula. In such a refinement, the coupling element may a line.

In a refinement, the barrier is coupled to the sleeve by a coupling element selected from the group consisting of a string, a line, a wire and combinations thereof.

In a refinement, one of the first and second substances is a solvent and the other of the first and second substances is selected from the group consisting of glucagon, epinephrine, heart attack rescue reagents, drugs, solid compounds, anti-venoms, antibodies, powdered compounds, liquid compounds, vitamins, nucleic acids, proteins, peptides, and combinations thereof.

In a refinement, the device comprises at least one housing in which the first and second chambers are disposed. The at least one housing comprises a window for exposing the barrier,

In a refinement, the barrier may be a plug.

In a refinement, the sleeve is coupled to a lower housing. The plug is disposed between the first and second chambers in an upper housing. The lower and upper housings are slidably and telescopically coupled together, wherein movement of the sleeve and lower housing with respect to the upper housing causing dislodgment of the plug and allowing the first and second substances to mix.

Methods for mixing at least two substances to form a mixture and for delivering the mixture to a patient are also disclosed. One disclosed method comprises providing a device in accordance with claim 1 wherein the dispense outlet is a cannula, moving the sleeve under manual pressure causing the barrier to become dislodged and the first and second substances to mix and form a mixture in at least one of the first and second chambers, and injecting the mixture into the patient through the cannula.

Other advantages and features will be apparent from the following detailed description when read in conjunction with the attached drawings.

### BRIEF DESCRIPTION OF DRAWINGS

For a more complete understanding of the disclosed methods and apparatuses, reference should be made to the embodiments illustrated in greater detail in the accompanying drawings, wherein:

FIG. 1 is a sectional and perspective view of a disclosed device for mixing two substances and delivering the mixture in the form of an injection;

FIG. 2 is another sectional perspective view of the device shown in FIG. 1 illustrating the removal of the outer sleeve which applies tension to the barrier between the two chambers by way of the outer sleeve being connected, coupled or tethered to the barrier by a string, line or other thin coupling element;

FIG. 3 is another perspective and sectional view of the device illustrated in FIGS. 1-2, particularly illustrating the sleeve and string detached from the barrier layer thereby enabling mixing of the substances into two chambers;

FIG. 4 is another perspective and sectional view of the device illustrated in FIGS. 1-3 without the sleeve;

FIG. 5 is another perspective and sectional view illustrating the compression of the spring and lower housing telescopically into the upper housing and may or may not prime the needle;

FIG. 6 is another perspective and sectional view of the device illustrated in FIGS. 1-5 illustrating the compression of the upper housing telescopically over the lower housing as the device is ready to inject the mixture into a patient;

FIG. 7 is another sectional perspective view of the device illustrated in FIGS. 1-6 with the injection progress;

FIG. 8 is another sectional perspective view of the device illustrated in FIGS. 1-7 with the injection completed;

FIG. 9 illustrates a spent or used device as disclosed in FIGS. 1-8 with the needle retracted back into the lower housing and ready for recycling or discard;

FIG. 10 is a perspective view of yet another disclosed device for mixing two substances injecting the mixture into a patient;

FIG. 11 is a perspective and exploded view of the device illustrated in FIG. 10;

FIG. 12 is another perspective and exploded view of the device illustrated in FIGS. 10-11;

FIG. 13 is a partial perspective and exploded view of the device illustrated in FIGS. 10-12;

FIG. 14 is another partial and perspective view of the device illustrated in FIGS. 10-13;

FIG. 15 is a front a sectional view illustrating yet another disclosed device that includes a dislodgeable stopper that prevents premature mixing of the two substances and the use of two chambers for the liquid substance disposed on either side of a chamber for a powdered substance;

FIG. 16 is another front sectional view of the device illustrated in FIG. 15;

FIG. 17 is yet another front sectional of the device illustrated in FIGS. 16-17;

FIG. 18 is a front sectional view of a disclosed device which does not include two separate chambers for mixing two substances, but which provides a rapid means for injecting a solution into a patient experiencing an emergency;

FIG. 19 is another front sectional view of the device illustrated in FIG. 18 at the beginning of an injection;

FIG. 20 is another front sectional view of the device illustrated in FIG. 18 at the beginning of an injection;

FIG. 21 is another front sectional view of the device illustrated in FIGS. 18-20 with the injection in progress;

FIG. 22 is another front sectional view of the device illustrated in FIGS. 18-21 as an injection is completed;

FIG. 23 is a bottom perspective view of the device illustrated in FIGS. 18-22; and

FIG. 24 is another bottom perspective view of the device illustrated in FIGS. 18-23.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION OF THE PRESENTLY PREFERRED EMBODIMENTS

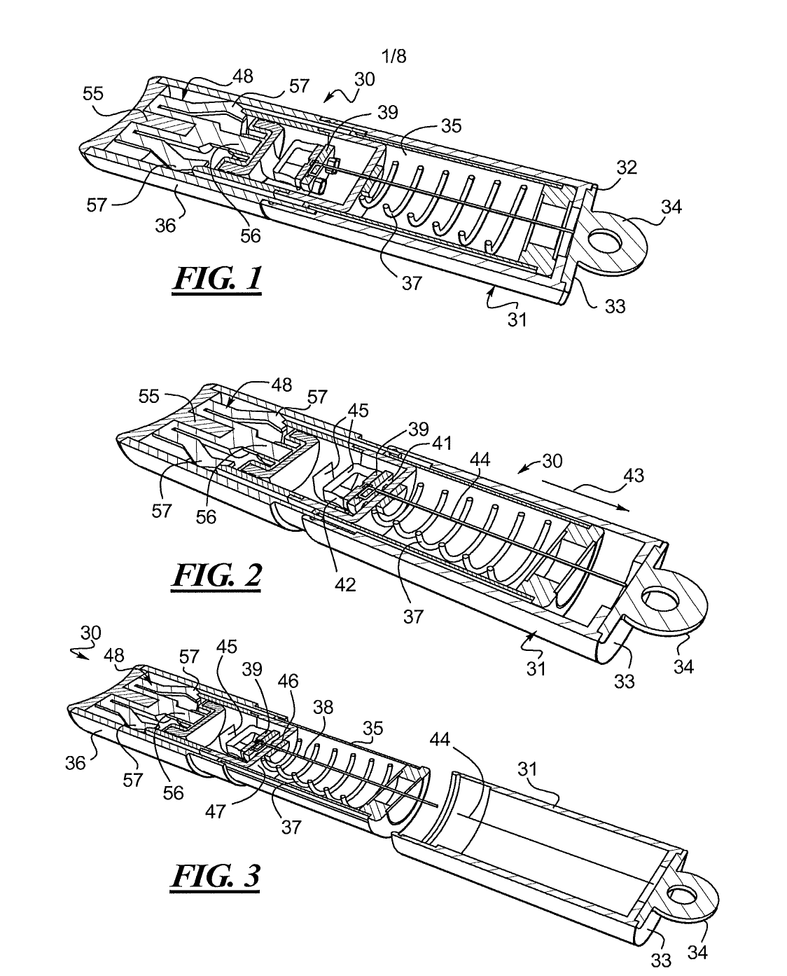

Turning first to FIGS. 1-9, the device 30 is disclosed which comprises an outer sleeve or cap 31 that may include an open end 32 covered by an end cap 33 that, in this example, includes an eyelet 34 that facilitates removal of the sleeve 31 from the lower and upper housings 35, 36. The lower housing 35 accommodates a spring or biasing member 37 and a needle or cannula 38. In the embodiment illustrated in FIGS. 1-9, the end cap 33 and sleeve 31 are coupled to or otherwise connected to a barrier 39. The barrier 39 separates a first chamber 41 from a second chamber 42. In one embodiment, the first chamber 41 accommodates a liquid, such as saline, and the second chamber 42 accommodates a powdered material, such as glucagon, antibodies, or a vaccine. Other examples will be apparent to those skilled in the art and are too numerous to mention here.

FIG. 2 illustrates the sleeve 31 and end cap 33 being pulled in the direction of the arrow 43. As illustrated in FIGS. 2 and 3, the end cap 33 is coupled to the barrier 39 by the coupling element 44 which, in this case, is a string or line. As the sleeve 31 and end cap 33 are pulled in the direction of the arrow 43, stress is imposed on the barrier 39 by the coupling element 44. In one embodiment, the barrier 39 comprises a piece of foil that may be torn or another suitable barrier material susceptible to tearing or rupture. When the foil barrier 39 is torn, powder in the second chamber 42 is exposed to liquid in the first chamber 41 and the mixing begins. Additional mixing element(s) 45 may be provided to enhance the mixing of the powder and liquid from the chambers 42 and 41 into a homogeneous mixture suitable for injection. In one embodiment, the mixing element(s) may be in the form of a film, such as a polymer film.

As illustrated in FIG. 3, the cannula 38 may provide a convenient path for the coupling element 44 to extend from the end cap 33 to the barrier 39. Both the cannula 38 and string or coupling element 44 extend through the membrane 46 that is supported by the cross frame 47.

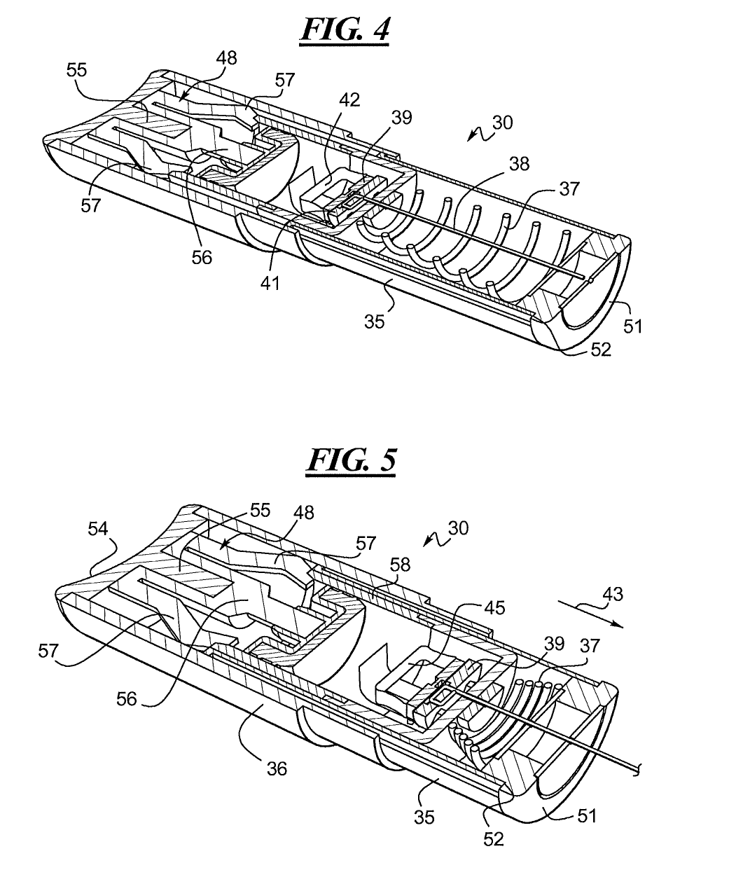

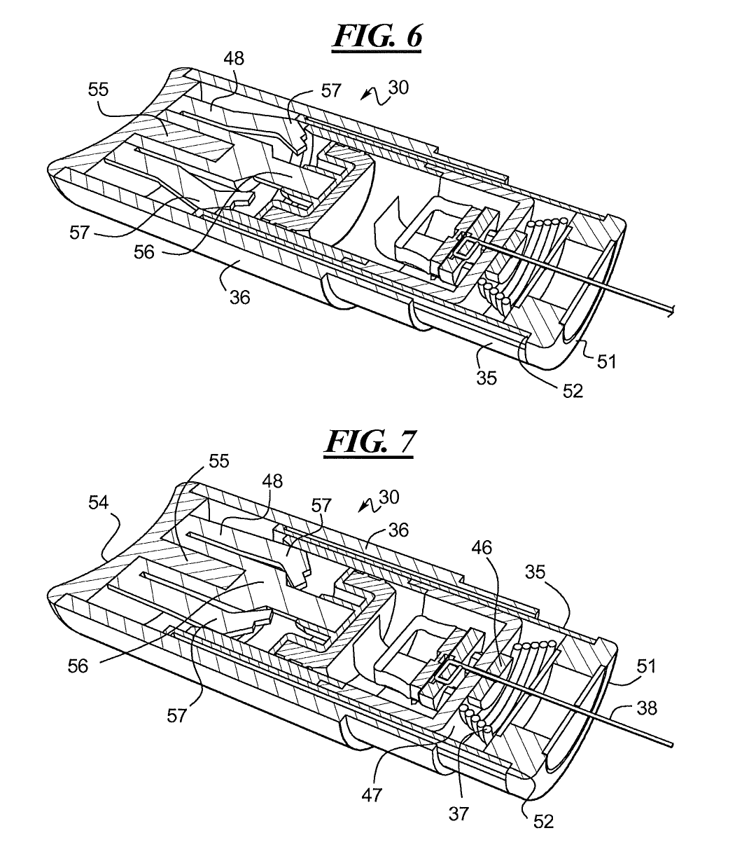

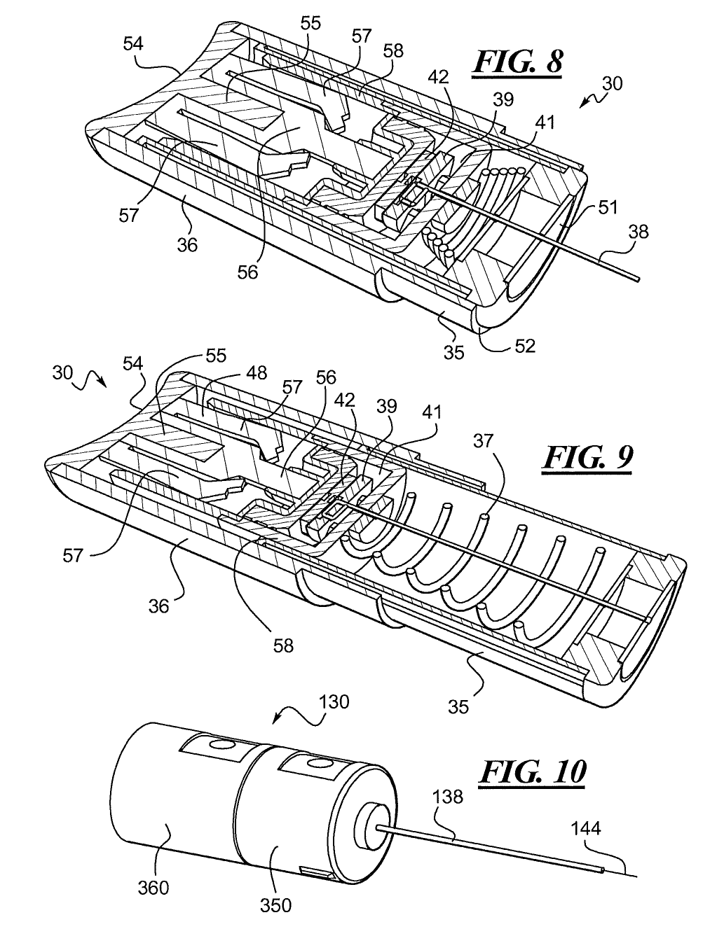

FIG. 4 illustrates the device 30 with the sleeve 31 and coupling element 44 removed and the barrier 39 damaged, torn or ruptures so that materials from the chambers 41 and 42 are being mixed. The mixture of materials from the chambers 41, 42 is now ready to be injected. FIG. 5 illustrates the compression of the lower housing 35 into the upper housing 36 and the compression of the spring 37. FIG. 6 illustrates the compression of the upper housing 36 over the lower housing 35 and the compression or loading of the secondary biasing member 48. In FIG. 6, the device 30 is fully loaded and ready for an injection which is illustrated in FIGS. 7-8. The secondary biasing member 48 is connected to the second chamber 36 by the end wall 54 which includes a post 55 that is frictionally received by the center post 56 of the secondary biasing member 48. As the device 30 is compressed into the position illustrated in FIG. 5, the outer legs 57 of the biasing member 48 become loaded for purposes of biasing the central housing 58 and lower housing 35 in the direction of the arrow 43. This action is illustrated sequentially in FIGS. 6-8. In FIG. 9, the biasing elements 37, 48 are fully extended and the cannula 38 has become retracted within the lower housing 35. FIGS. 5-9 also illustrate the end plug 51 which covers the open end 52 of the lower housing 35. Preferably, the end plug 51 is equipped with a membrane through which the cannula 38 passes.

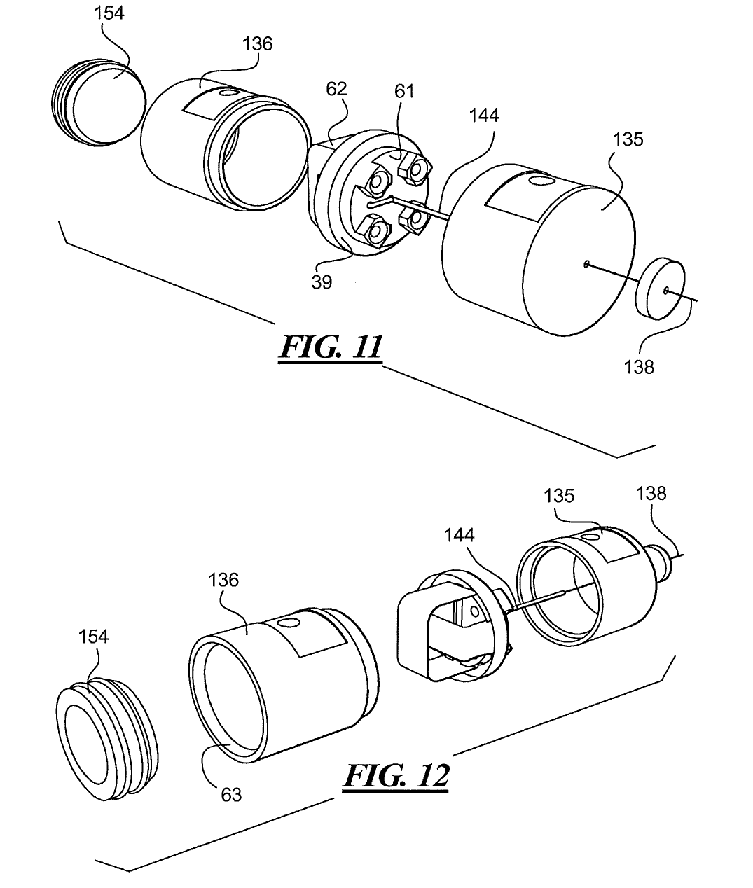

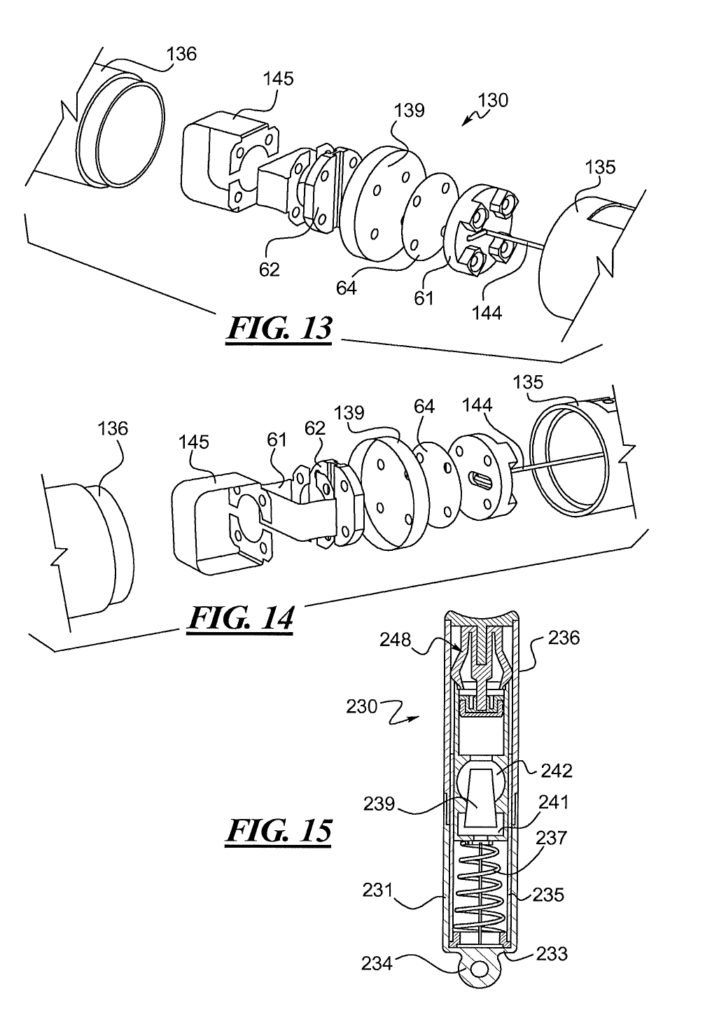

FIGS. 10-14 illustrate yet another embodiment 130. The device 130 includes an upper housing 136, lower housing 135 and cannula 138. As illustrated in FIG. 11, a coupling element 144 is connected to the barrier 139. In the embodiment 130 illustrated in FIGS. 10-14, the barrier 139 includes a pair of plates 61, 62 for purposes of imposing a shear force on the foil barrier 139. The open end 63 of the upper housing 136 is covered by the end plug 154. Mixing elements 145 may also be provided to move powder from above the foil barrier 139 two below the foil barrier 139 or towards the right in the orientation of FIGS. 13 and 14. The foil barrier 139 may be equipped with the plates 61, 62 as discussed above and an additional one or more polycarbonate films 64 (FIGS. 13-14) may be included for purposes of allowing the foil barrier 139 and foil plates 61, 62 to flex.

In operation, the coupling element 144 may be coupled to a protective sleeve or cap (not shown). Tension applied to the coupling element 144 will be translated to the barrier 139, which eventually tears or ruptures. The powder in the second chamber disposed opposite the barrier 139 from the lower housing 135 is then mixed with liquid disposed in the first chamber disposed opposite the barrier 139 from the upper housing 136.

As shown in FIGS. 15-17, a coupling element 44 (FIG. 1-9) or 144 (FIGS. 10-14) extending between a removable item such as a sleeve or cap 31 or other handle is not necessary. For example, any type of movable or disruptable barrier disposed between the first and second chambers 41, 42 or 241, 242 may be employed. In FIGS. 15-17, the movable barrier is provided in the form of a plug 239. While the two embodiments illustrated in FIGS. 1-9 and 10-14 disclose foil barriers 39, 139 other impermeable or relatively impermeable barriers will be apparent to those skilled in the art including, but not limited to, other metallic barriers or membranes and non-metallic barriers or membranes such as polymer barriers or membranes.

Turning to FIGS. 15-17, an additional device to 30 is illustrated which includes a conical plug 239 that serves as a barrier between the first chamber 241 and second chamber 242. As seen in the sequence illustrated in FIGS. 15-17, as the device 230 is compressed, the plug 239 extends upward towards the secondary biasing element 248 thereby establishing communication between the lower and second chambers 241, 242. Otherwise the device 230 is similar in construction to the device 30 illustrated in FIGS. 1-9 as it includes a spring 237, upper and lower housings 236, 235, an end cap 233, sleeve 231, eyelet 234 and secondary biasing member 248.

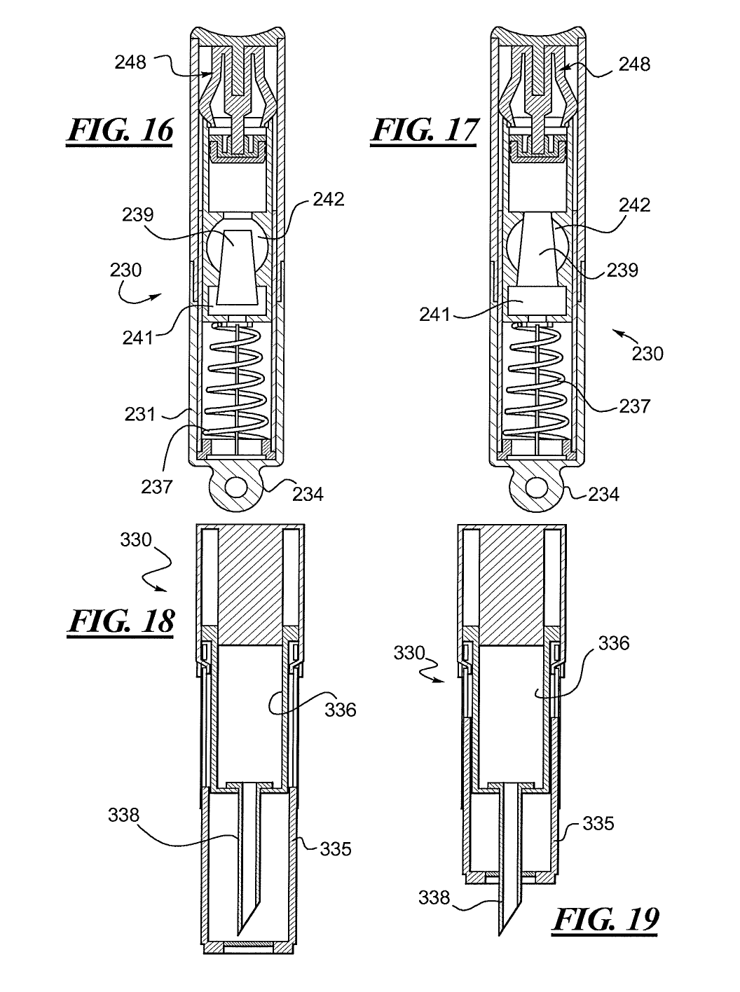

While FIGS. 15-17 illustrate a conical plug 239, the shape of the plug to 39 is relatively unimportant. Dislodgment of the plug from its position between the first and second chambers 241, 242 (compare FIGS. 16 and 17) upon the application of manual pressure is a primary function of the plug 239. For that matter, the application of manual pressure can be used to terror, damage or remove either one of the foil barriers 39, 139 disclosed above in FIGS. 1-14.

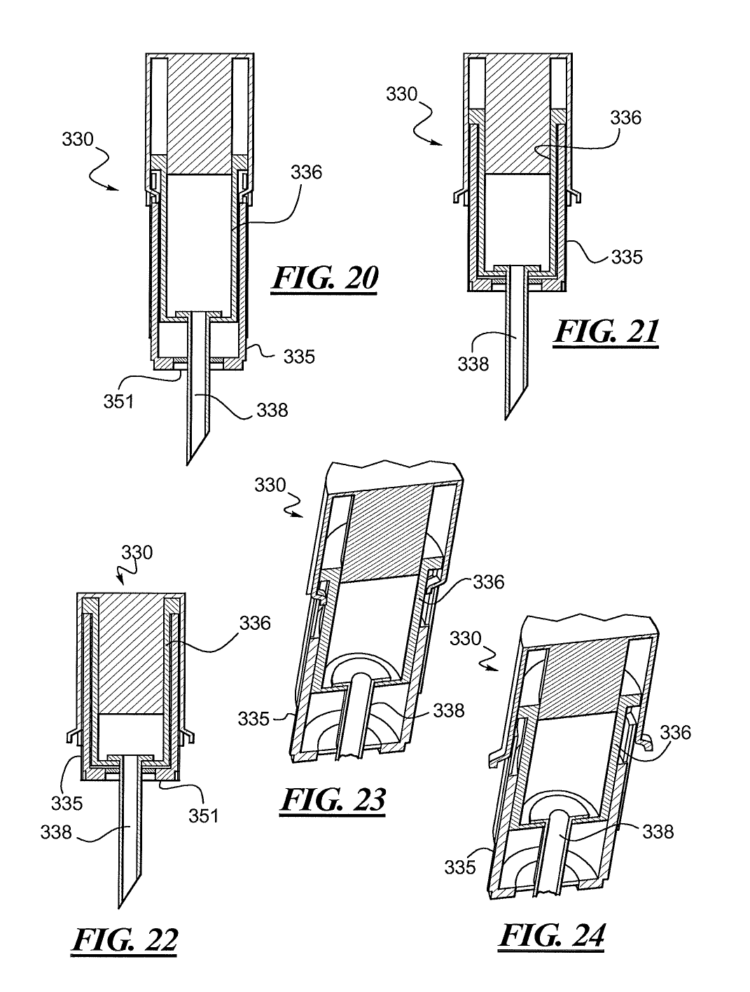

FIGS. 18-24 illustrate a single chamber device 330 which includes a lower housing 335 and an upper housing 336. The upper housing 136 includes the single chamber in communication with the cannula 338. As the device 330 is compressed, the cannula extends through the end cap 351 for injecting as illustrated sequentially in FIGS. 19-22. The embodiment illustrated in FIGS. 18-24th is intended for applications where mixing of two different substances is not necessary.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

What is claimed is:

1. A device for mixing at least two substances to form a mixture and for delivering the mixture to a human or animal body, the device comprising:
a first chamber for containing a first substance;
a second chamber for containing a second substance;
a barrier comprising a plug disposed between the first and second chambers to isolate the first chamber from the second chamber and prevent the first substance from mixing with the second substance until such mixing is desired, wherein the barrier is susceptible to tearing or rupture, causing the first and second substances to mix in at least one of the first chamber or the second chamber to form the mixture; and
a cannula in fluid communication with the first chamber for delivering the mixture into the patient.

2. A device as in claim 1, wherein the cannula comprises a needle.

3. A device as in claim 1, wherein the second substance is a powder.

4. A device as in claim 3, wherein the first substance is a liquid.

5. A device as in claim 1, wherein the first substance is a liquid.

6. A device as in claim 1, further comprising:
an outer sleeve;
an upper housing; and
a lower housing,
wherein the first chamber is disposed between the sleeve and the second chamber.

7. A device as in claim 6, wherein the second chamber is disposed between the upper housing and the barrier.

8. A device as in claim 1, wherein the barrier comprises a piece of foil.

9. A device as in claim 8, wherein the barrier is coupled to a sleeve by a coupling element.

10. A device as in claim 9, wherein the coupling element comprises a line.

11. A device as in claim 10, wherein the line passes through the cannula.

12. A device as in claim 1, wherein the barrier is coupled to a sleeve by a coupling element that passes through the cannula, and wherein movement of the sleeve under manual pressure tears or ruptures the barrier.

13. A device as in claim 1, wherein the barrier is coupled to a sleeve by a coupling element selected from the group consisting of a string, a line, a wire and combinations thereof.

14. A device as in claim 1, wherein one of the first and second substances is a solvent and the other of the first and second substances is selected from the group consisting of glucagon, epinephrine, heart attack rescue reagents, drugs, solid compounds, powdered compounds, liquid compounds, lipids, sugars, steroids, hormones, vitamins, nucleic acids, vaccines, anti-venoms, antibodies, proteins, peptides, nano-particles and combinations thereof.

15. A device as in claim 1, further comprising at least one housing in which the first and second chambers are disposed, the at least one housing comprising a window for exposing the barrier.

16. A device as in claim 1, further comprising:
an outer sleeve;
an upper housing; and
a lower housing,
wherein the sleeve is coupled to the lower housing, wherein the plug is disposed between the first and second chambers in the upper housing, wherein the lower and upper housings are slidably and telescopically coupled together, and wherein movement of the sleeve and lower housing with respect to the upper housing causes dislodgment of the plug and allows the first and second substances to mix.

17. A device for injecting a substance into a human or animal body, the device comprising:
a first housing;
a second housing slidably and telescopically coupled with the first housing;
a chamber disposed in at least one of the first housing or the second housing; and
a needle in fluid communication with the chamber for injecting the mixture into the human or animal body,
wherein the first and second housings are configured to telescope from a first configuration, in which the needle is completely housed within at least one of the housings, to a second configuration, in which the needle is extended out of the housings for injecting the substance, to a third configuration, in which the substance is advanced out of the needle, and to a fourth configuration, in which the needle is refracted back into at least one of the housings.

18. A device as in claim 17, wherein the first housing telescopes into the second housing, and wherein the device further comprises a sleeve disposed over at least a portion of the first housing.

19. A device as in claim 17, further comprising a removable sleeve covering an end of the device through which the needle extends

20. A device as in claim 17, further comprising a spring configured to compress when the needle extends out of the housing and spring back to an uncompressed configuration to retract the needle into at least one of the housings.

21. A device as in claim 17, further comprising an end cap through which the needle pierces to extend out of the housings.

22. A device as in claim 17, wherein the chamber is disposed in the first housing, and wherein the first housing telescopes at least partially into the second housing to advance the needle through an opening in the second housing.

23. A device as in claim 17, wherein the chamber is disposed in the first housing, and wherein the second housing telescopes at least partially into the first housing to advance the needle through an opening in the second housing.

24. A device as in claim 23, further comprising a sleeve removably disposed over the second housing to cover the opening before use of the device.

25. A device as in claim 24, further comprising:
a barrier dividing the chamber into two parts; and
a line connected with the sleeve at one end and extending through the cannula to connect to the barrier,
wherein removing the sleeve from the second housing causes the line to tear or rupture the barrier, thus allowing two different substances disposed in the two parts of the chamber to form a mixture, wherein the mixture is injected into the body via the needle.

26. A device as in claim 17, further comprising the substance, disposed in the chamber, wherein the substance is selected from the group consisting of glucagon, epinephrine, heart attack rescue reagents, drugs, solid compounds, powdered compounds, liquid compounds, lipids, sugars, steroids, hormones, vitamins, nucleic acids, vaccines, anti-venoms, antibodies, proteins, peptides, nano-particles and combinations thereof.
